(19) United States
(12) Patent Application Publication
(10) Pub. No.: US 2006/0148865 A1
(43) Pub. Date: Jul. 6, 2006

(54) Preparation of chemical compounds

(76) Inventors: Michael Tolar Martin, Durham, NC (US); John Charles Roberts, Durham, NC (US); Jennifer Fell Toczko, Durham, NC (US)

Correspondence Address: GLAXOSMITHKLINE;CORPORATE INTELLECTUAL PROPERTY, MAI B475, FIVE MOORE DR., PO BOX 13398, RESEARCH TRIANGLE PARK, NC, 27709-3398, US

(21) Appl. No.: 10/560,500

(22) PCT Filed: Jun. 25, 2004
(86) PCT No.: PCT/US04/20353

§ 371 Date: Dec. 12, 2005

Related U.S. Application Data
(60) Provisional application No. 60/483,002, filed on Jun. 27, 2003.
(60) Provisional application No. 60/483,002, filed on Jun. 27, 2003.

(51) Int. Cl. A61K 31/427; C07D 417/14

(52) U.S. Cl. 514/365; 548/181

(57) ABSTRACT

The present invention is directed to processes for the preparation of N-(3R, 3aS, 6aR)-hexahydrofuro[2,3-b]furan-3-yl-oxycarbonyl-, (4S,5R)-4-[4-(2-methylthiazolo-4-methyloxy)-benzyl]-5-i-butyl-[(3,4-methylenedioxyphenyl)sulfonyl]-aminomethyl-2,2-dimethyl-oxazolidine.

### BACKGROUND OF THE INVENTION

The human immunodeficiency virus (“HIV”) is the causative agent of acquired immunodeficiency syndrome (“AIDS”), a disease characterized by the destruction of the immune system, particularly of CD4⁺ T-cells, with attendant susceptibility to opportunistic infections, and its precursor AIDS-related complex (“ARC”), a syndrome characterized by symptoms such as persistent generalized lymphadenopathy, fever and weight loss.

Among the drugs currently used to treat HIV infections in humans are those that inhibit the HIV aspartyl protease enzyme. Drugs that are used as protease inhibitors are, in general, chemically complex and are difficult to prepare in a cost-effective and efficient manner. As a result of the inherent complexity of these molecules, new and more efficient methods for their preparation are of value.

N-(3R, 3aS, 6aR)-hexahydrofuro[2,3-b]furan-3-yl-oxycarbonyl-, (4S,5R)-4-[4-(2-methylthiazolo-4-methyloxy)-benzyl]-5-1-butyl-[(3,4-methylenedioxyphenyl)sulfonyl]-aminomethyl-2,2-dimethyl-oxazolidine, described in WO 00/76961, is a compound of interest as a new therapeutic agent in the treatment of HIV infections and associated conditions. There exists a need to produce this compound in large quantities for clinical investigation of the safety and efficacy of the compound in therapy.

The present invention concern processes for the preparation of N-(3R, 3aS, 6aR)-hexahydrofuro[2,3-b]furan-3-yl-oxycarbonyl-, (4S,5R)-4-[4-(2-methylthiazolo-4-methyloxy)-benzyl]-5-i-butyl-[(3,4-methylenedioxyphenyl)sulfonyl]-aminomethyl-2,2-dimethyl-oxazolidine.

The present invention also features chemical compounds useful as intermediates in the preparation of compounds that may function as inhibitors of HIV aspartyl protease.

WO 00/76961 discloses processes that could be applied to the preparation of N-(3R, 3aS, 6aR)-hexahydrofaro[2,3-b]furan-3-yl-oxycarbonyl-, (4S,5R)-4-[4-(2-methylthiazolo-4-methyloxy)-benzyl]-5-i-butyl-[(3,4-methylenedioxyphenyl)sulfonyl]-aminomethyl-2,2-dimethyl-oxazolidine. These processes are not desirable for large-scale manufacture due to low throughput, slow filtrations, variable purity of intermediates and product, and potential difficulties with removal of intermediates from reactors, long drying times, and reproducibility. Processes of the present invention reduce the number of operations and isolations, and are efficient, safe, and reproducible, thereby rendering the processes conducive to use in large-scale manufacture of N-(3R, 3aS, 6aR)-hexahydrofuro[2,3-b]furan-3-yl-oxycarbonyl-, (4S,5R)-4-[4-(2-methylthiazolo-4-methyloxy)-benzyl]-5-i-butyl-[(3,4-methylenedioxyphenyl)sulfonyl]-aminomethyl-2,2-dimethyl-oxazolidine.

### BRIEF DESCRIPTION OF THE INVENTION

The present invention provides processes and compounds that are useful in the preparation of N-(3R, 3aS, 6aR)-hexahydrofaro[2,3-b]furan-3-yl-oxycarbonyl-, (4S,5R)-4-[4-(2-methylthiazolo-4-methyloxy)-benzyl]-5-i-butyl-[(3,4-methylenedioxyphenyl)sulfonyl]-aminomethyl-2,2-dimethyl-oxazolidine.

### DETAILED DESCRIPTION OF THE INVENTION

The present invention provides processes and compounds that are useful in the preparation of N-(3R, 3aS, 6aR)-hexahydrofuro[2,3-b]furan-3-yl-oxycarbonyl-, (4S,5R)-4-[4-(2-methylthiazolo-methyloxy)-benzyl]-5-i-butyl-[(3,4-methylenedioxyphenyl)sulfonyl]-aminomethyl-2,2-dimethyl-oxazolidine, a compound of formula (I).

The present invention features a process for the preparation of a compound of formula (1) 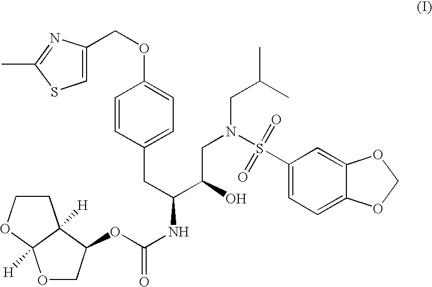
comprising

(a) treating a compound of formula (II)

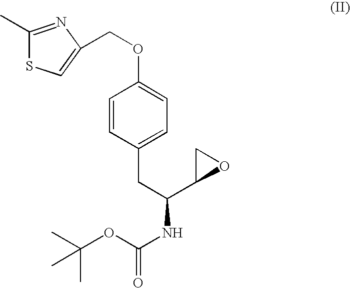

with excess isobutylamine in an alcohol-containing solvent to form a compound of formula (III);

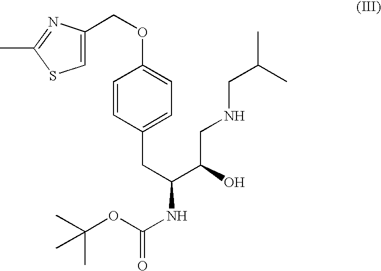

(b) treating a compound of formula (II) with a compound of formula (IV) 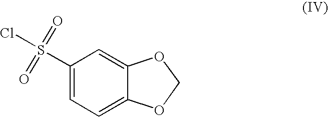
in the presence of an aqueous base to form a compound of formula (V);

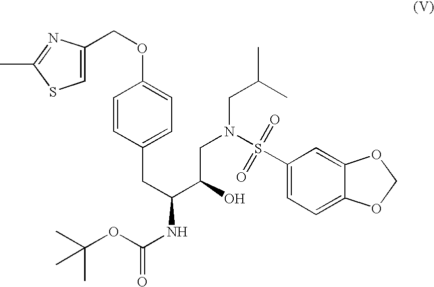

(c) deprotecting a compound of formula (V) to form a compound of formula (VI)

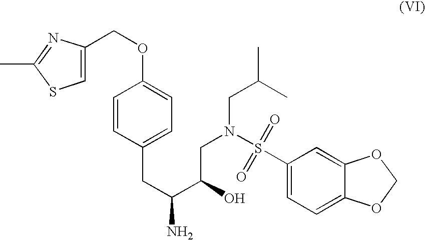

(d) and coupling with a compound of formula (VII) 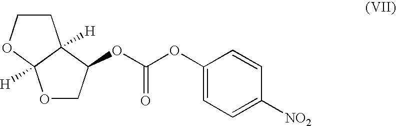
to form a compound of formula (I).

Alternatively, step (d) may be performed by coupling with a compound of formula (VIII) 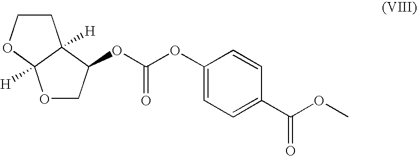
to form a compound of formula (I).

Alternatively, step (d) may be performed by coupling with a compound of formula (IX) 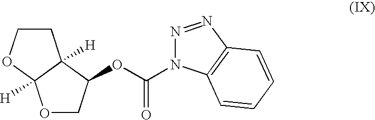
to form a compound of formula (I).

Alternatively, step (b) may be performed in the presence of a non-aqueous base.

Optionally, the product of step (d) may be crystallized by treatment in an appropriate solvent, for example, isopropyl alcohol-water.

The present invention also features a process for the preparation of a compound of formula (I) comprising steps (a), (b), (c) and (d) above wherein steps (a) and (b) are combined in a one-pot reaction to yield a compound of formula (V) which is isolated and in which steps (c) and (d) are combined in a one-pot reaction to yield a compound of formula (I) via a compound of formula (VI).

The combination of steps (a) and (b) in a one-pot process may be critical to the efficiency of the present invention. The standard method of conducting step (a), in refluxing ethanol or isopropanol, was not conducive to its combination with step (b) due to reduced inefficiencies associated with the necessity to exchange of all of the reaction solvent. Very high temperatures necessary for the execution of this reaction in non-alcoholic solvents, e.g. in toluene, is not advisable due to the stability limits of a compound of formula (II). It was found, however, that a small amount of methanol accelerates step (a) even when diluted with acetonitrile, a solvent with favorable properties for the azeotropic removal of methanol and this observation was exploited so that steps (a) and steps (b) could be combined.

The combination of steps (c) and (d) in a one-pot process may be critical to the efficiency of the present invention. The solvent system of tetrahydrofuran-water was identified as one which could accomplish all of the following: 1) solubilize a compound of formula (V) and the methane sulfonic acid salt thereof; 2) solubilize a compound of formula (VI) and the methane sulfonic acid salts thereof; 3) be used as a medium for step (c) 4) be used as a medium for step (d); 5) solubilize a compound of formula (I); and 6) be modified for an aqueous workup in such a way that a solvent exchange to the crystallization solvent (isopropanol-water) could be accomplished efficiently.

The following abbreviations may be used in the specification:
g (grams);
L (liters);
μL (microliters);
mM (millimolar);
mmol (millimoles);
min (minutes);
mp (melting point);
MeOH (methanol);
THF (tetrahydrofuran);
HOAc (acetic acid);
CBZ (benzyloxycarbonyl);
DMAP (4-dimethylaminopyridine);
DIBAL (di-isobutylaluminum hydride);
MsOH (methane sulfonic acid)
Pd/C (palladium on carbon)
LC (liquid chromatography)
GC (gas chromatography)
MTBE (tert-butyl methyl ether)
IPAC (isopropyl acetate)
DCM (dichloromethane)
mg (milligrams);
mL (milliliters);
M (molar);
mol (moles);
rt (room temperature);
h (hours);
TLC (thin layer chromatography);
TEA (triethylamine);
AcOEt (ethyl acetate);
BOC (tert-butyloxycarbonyl);
Ac (acetyl);
NBS N-bromosuccinimide);
LAH (lithium aluminum hydride);

Step (a) may be carried out by reacting tert-butyl (1S)-2-{4-[(2-methyl-1,3-thiazol-4-yl)methoxy]phenyl}-1-[(2S)-oxiran-2-yl]ethylcarbamate with an amine, preferably isobutylamine, in the presence of a suitable solvent, preferably acetonitrile and methanol. The reaction is advantageously carried out at (or near) reflux. The product of step (a), a compound of formula (III), may be isolated or taken directly to step (b).

Step (b) may be carried out by addition of a sulfonyl chloride, preferably 1,3-benzodioxole-5-sulfonyl chloride (Commercial supplier: SF-Chem P.O. Box 1964 CH-4133 Pratteln 1 Switzerland) in a suitable solvent, preferably acetonitrile, while maintaining 25° C. with non-aqueous base, preferably N-methylmorpholine, present during the addition. If aqueous base, preferably sodium bicarbonate, is used, it is added after the sulfonyl chloride addition while maintaining a temperature of about 25° C. The product of the reaction, a compound of formula (V), is crystallized in a suitable solvent, preferably acetonitrile-water.

Step (c) may be carried out by deprotection of a compound of formula (V) by treatment with an acid, preferably methane sulfonic acid, in a suitable solvent, preferably THF-water.

Step (d) may be achieved by neutralization of the acid used in step (c) with a base, preferably triethylamine, treatment of the free-based, deprotected compound of formula (VII) [(3R,3aS,6aR)-hexahydrofuro[2,3-b]furan-3-yl 4-nitrophenyl carbonate], and heating at (or near) reflux. Compounds of formula (VIII) [(3R,3 aS,6aR)-hexahydrofuro[2,3-b]furan-3-yl-4-carbomethoxyphenyl carbonate], and (IX) [(3R,3aS,6aR)-hexahydrofuro[2,3-b]furan-3-yl-1N-benzotriazole carbamate] may also be used. After an aqueous workup, the product solution may be exchanged for a suitable solvent, preferably isopropanol-water, and crystallized.

A compound of formula (II) may be made by treating tert-butyl (15)-2-[4-(benzyloxy)phenyl]-1-[(2S)-oxiran-2-yl]ethylcarbamate (Commercial supplier: Aerojet Fine Chemicals P.O. Box 1718 Rancho Cordova, Calif. 95741) with a hydrogenation catalyst, preferably palladium on carbon, a hydrogen source, preferably hydrogen gas, in a suitable solvent, preferably tetrahydrofuran at ˜25° C.: The catalyst may be removed by filtration and an alkylating agent, preferably 4-(chloromethyl)-2-methyl-1,3-thiazole hydrochloride (Commercial supplier: Lancaster Synthesis Inc., P.O. Box 1000, Windham N.H. 03087-9977), may be added followed by a source of iodide, preferably sodium iodide, and a base, preferably sodium tert-butoxide while maintaining a temperature in the range of 30-40° C. Finally a solution of aqueous base, preferably sodium hydroxide, is added to close any epoxide inadvertently opened by the iodide anion. The resulting solution may be washed with aqueous solutions, preferably pure water and sodium chloride solution, and solvent exchanged to an appropriate crystallization solvent, preferably heptane-ethyl acetate.

A compound of formula (VII) may be made from (3R,3aS,6aR)-hexahydrofuro[2,3-b]furan-3-ol by treatment in a suitable solvent, preferably DCM-IPAC, with a base, preferably pyridine, and a 4-nitrophenoxy carbonyl source, preferably 4-nitrophenyl chloroformate. The product compound may be isolated subsequent to aqueous washes, preferably with dilute hydrochloric acid and then sodium bicarbonate solution, by solvent exchange into a suitable crystallization solvent, preferably isopropyl acetate, and crystallization.

A compound of formula (VIII) may be made from (3R,3aS,6aR)-hexahydrofuro[2,3-b]furan-3-ol by treatment in a suitable solvent, with a base, preferably pyridine, and 4-carbomethoxyphenyl chloroformate. The product compound may be isolated subsequent to aqueous washes, preferably with dilute hydrochloric acid and then sodium chloride solution, by solvent exchange into a suitable crystallization solvent, preferably ethyl acetate, and crystallization.

A compound of formula (IX) may be made from (3R,3aS,6aR)-hexahydrofuro[2,3-b]furan-3-ol by treatment in a suitable solvent, with N-methylmorpholine and 1H-1,2,3-benzotriazole-1-carbonyl chloride. The product compound may be isolated by crystallization.

The present invention features a process illustrated by Scheme I: 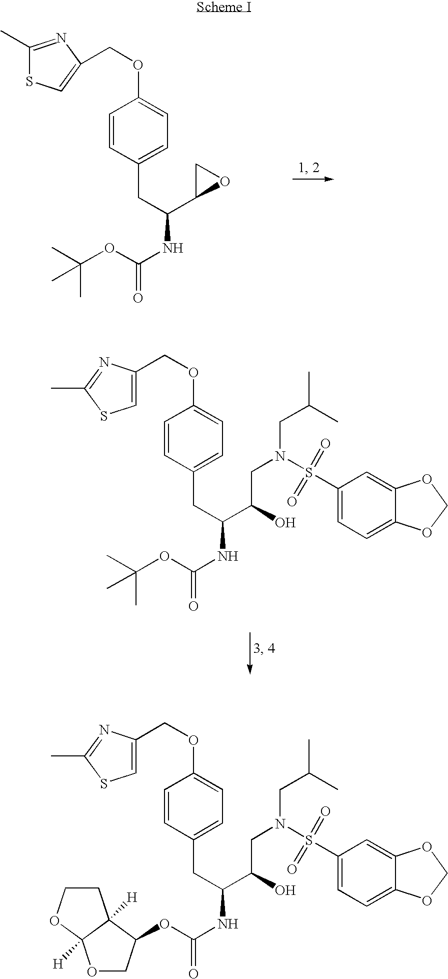
1. MeCN, isobutylamine, MeOH
2. MeCN, (IV), sodium bicarbonate (aq)
3. THF-water, MsOH
4. THF-water, TEA, (VII)
5. DCM-IPAC, (3R,3aS,6aR)-hexahydrofuro[2,3-b]furan-3-ol, 4-nitrophenyl chloroformate, pyridine

Reaction 2 of Scheme I may alternatively be MeCN, N-methylmorpholine, (IV).

The present invention features a process for the preparation of a compound of formula (I) 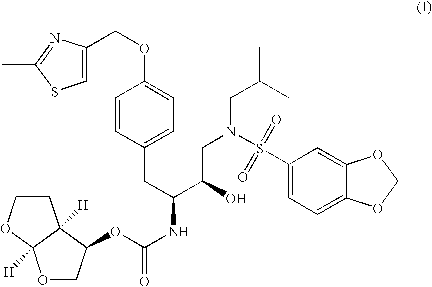
comprising:

(a) reacting a compound of formula (II)

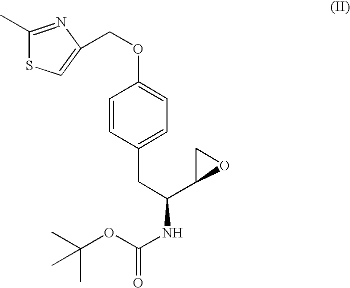

with excess isobutylamine in an alcohol-containing solvent to form a compound of formula (III);

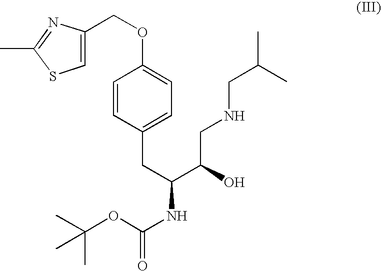

(b) reacting a compound of formula (III) with a compound of formula (IV) 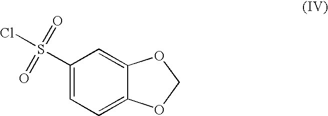
in the presence of an aqueous base to form a compound of formula (V);

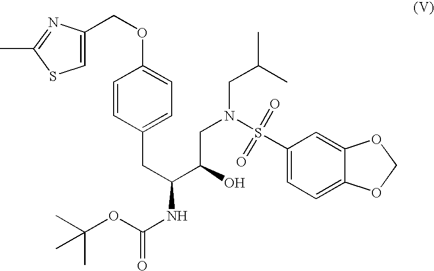

(c) deprotecting a compound of formula (V) and coupling with a compound of formula (VII) 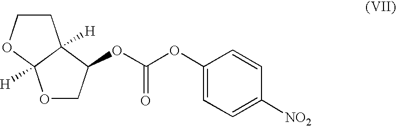
to form a compound of formula (I).

The present invention further features a process for the preparation of (3R,3aS,6aR)-hexahydrofuro[2,3-b]furan-3-yl 4-nitrophenyl carbonate of the formula 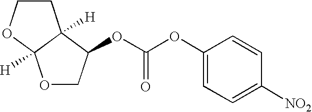
comprising reacting (3R,3aS,6aR)-hexahydrofuro[2,3-b]furan-3-ol) of the formula 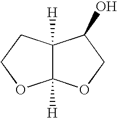
with 4-nitrophenyl chloroformate in a suitable solvent to form (3R,3aS,6aR)-hexahydrofuro[2,3-b]furan-3-yl 4-nitrophenyl carbonate.

The present invention features (3R,3aS,6aR)-hexahydrofuro[2,3-b]furan-3-yl 4-nitrophenyl carbonate of the formula 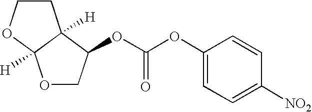
made by the process described above. The specific conditions may be critical to the efficiency of the process for preparing (3R,3aS,6aR)-hexahydrofuro[2,3-b]furan-3-yl 4-nitrophenyl carbonate. The use of isopropyl acetate for the isolation permits a very high tolerance to the purity of the (3R,3aS,6aR)-hexahydrofuro[2,3-b]furan-3-ol) input (e.g. 70-95% purity), affording >98% purity for the product [(3R,3aS,6aR)-hexahydrofuro[2,3-b]furan-3-yl 4-nitrophenyl carbonate] Furthermore, under the conditions exemplified, an inevitable byproduct, namely bis(4-nitrophenyl) carbonate, is both minimized during the reaction (to 10-15%) and substantially reduced (to <2%) during the recrystallization.

The following examples further describe and demonstrate particular embodiments within the scope of the present invention. The examples are given solely for illustration and are not to be construed as limitations as many variations are possible without departing from spirit and scope of the invention.

Unless otherwise noted, all starting materials were obtained from commercial suppliers and used without further purification.

Unless otherwise indicated, all temperatures are expressed in ° C. (degrees Centigrade). All reactions are conducted under an inert atmosphere at room temperature unless otherwise noted.

### EXAMPLE 1

### Preparation of tert-butyl (1S,2R)-3-[(1,3-benzodioxol-5-ylsulfonyl(isobutyl)amino]-2-hydroxy-1-{4-[(2-methyl-1,3-thiazol-4-yl)methoxy]benzyl}propylcarbamate

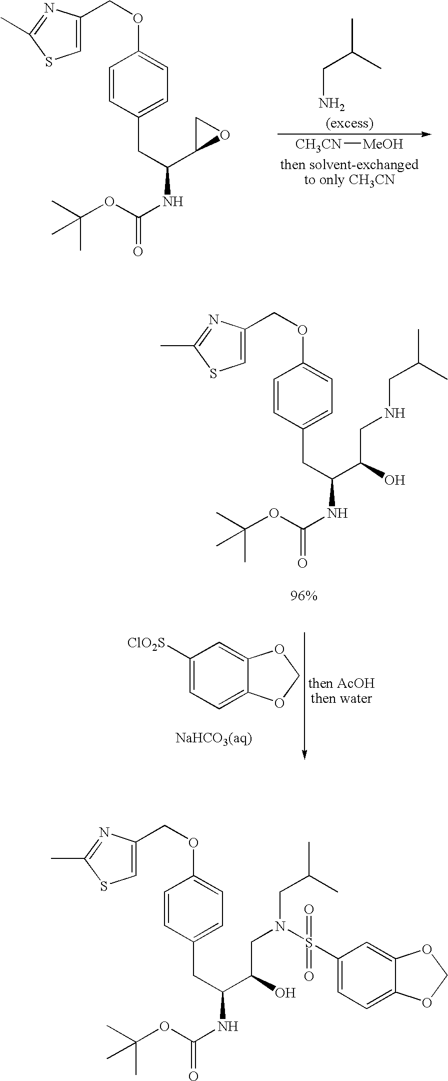

A reaction vessel was charged with tert-butyl (1S)-2-{4-[(2-methyl-1,3-thiazol-4-yl)methoxy]phenyl}-1-[(2S)-oxiran-2-yl]ethylcarbamate (1.0 wt.) followed by acetonitrile (3.5 vol.), methanol (1.0 vol.), and isobutylamine (8.3 equiv., 2.1 vol.). The resulting mixture was heated to reflux and held at reflux for 3 h. Acetonitrile (9.0 vol.) was charged and distillate (9.0 vol., 6.8 wt.) was collected at atmospheric pressure. A second portion of acetonitrile (9.0 vol.) was charged and distillate (9.0 vol., 6.9 wt.) was collected at atmospheric pressure. Acetonitrile (2.5 vol.) was charged and the reaction mixture was cooled to ˜25° C. A solution of 1,3-benzodioxole-5-sulfonyl chloride (1.1 equiv., 0.62 wt) in acetonitrile (2 vol.) was charged while maintaining a temperature of ˜25° C. A solution of sodium bicarbonate (1.2 equiv., 0.26 wt.) in water (2.3 vol.) was charged while maintaining a temperature of ˜25° C. The resulting reaction mixture was stirred for 4 h. Acetic acid (2.0 equiv., 0.29 vol.) was charged followed by water (10 vol.) and the resulting slurry was stirred for 1 h. The solids are filtered and washed with a solution of acetonitrile (2.5 vol.) in water (7.5 vol). The product was dried at ˜50° C. in a vacuum oven. The title product (1.5 wt, 90%) was a light beige solid.

### EXAMPLE 2

### Preparation of N-(3R, 3aS, 6aR)-hexahydrofuro[2,3-b]furan-3-yl-oxycarbonyl-(4S,5R)-4-[4-(2-methylthiazolo-4-meth loxy)-benzyl]-5-1-butyl-[(3,4-methylenedioxyphenyl)sulfonyl]-aminomethyl-2,2-dimethyl-oxazolidine

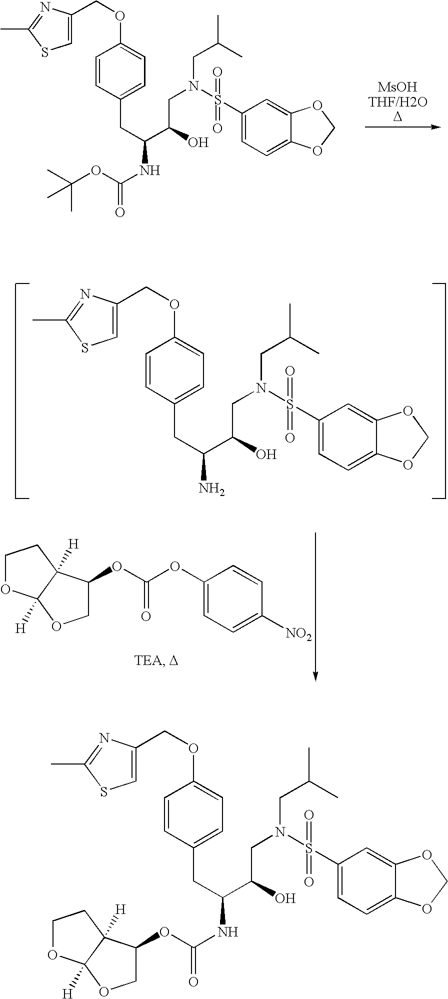 A reaction vessel was charged with tert-butyl (1S,2R)-3-[(1,3-benzodioxol-S-ylsulfonyl)(isobutyl)amino]-2-hydroxy-1-{4-[(2-methyl-1,3-thiazol-4-yl)methoxy]benzyl}propylcarbamate (1.0 wt.), tetrahydrofuran (5.0 vol.), and water (0.05 vol.) and stirred at ˜25° C. The reaction vessel was then charged with methane sulfonic acid (3.0 equiv., 0.30 vol.), and the resulting mixture was heated over 30 min to ˜50° C., stirred, and then heated over 30 min to reflux. Water (0.25 vol.) was added, the reaction mixture was cooled to ˜50° C., and triethylamine (3.7 equiv., 0.80 vol.) was added followed by solid (3R,3aS,6aR)-hexahydrofuro[2,3-b]furan-3-yl 4-nitrophenyl carbonate. (1.05 equiv., 0.48 wt.). The resulting mixture was brought to reflux, and stirred for 3.5 h. The reaction mixture was cooled to ˜50° C. and tert-butyl methyl ether (3.0 vol.) was added. Maintaining ˜50° C., the mixture was washed with water (2.1 vol.), 10% (by weight) aqueous potassium carbonate (2×2.1 vol., 1.0 equiv) and 5% (by weight) aqueous acetic acid (2.1 vol., 1.1 equiv) At atmospheric pressure, the organic mixture was concentrated to ˜4.2 vol (i.e. ˜3.3 vol removed) and diluted to the original volume with isopropyl alcohol (˜3.3 vol.) The mixture was again taken to 4.2 vol and diluted to the original volume with isopropyl alcohol (−3.3 vol.). After a final concentration to 4.2 vol, isopropyl alcohol (9.5 vol.) and water (0.30 vol) was charged. The resulting mixture heated to dissolve all solids, cooled to 50° C., seeded with N-(3R, 3aS, 6aR)-hexahydrofuro[2,3-b]furan-3-yl-oxycarbonyl-, (4S,5R)-4-[4-(2-methylthiazolo-4-methyloxy)-benzyl]-5-i-butyl-[(3,4-methylenedioxyphenyl)sulfonyl]-aminomethyl-2,2-dimethyl-oxazolidine crystals (0.01 wt), and held at 50° C. for 1 h (or until appreciable crystallization has occurred. The resulting slurry was cooled to room temperature at 0.7° C./min (˜35 min). The solid product was filtered, washed with 50:50 isopropyl alcohol/heptane (4 vol.) and heptane (4 vol.), and dried at ˜50° C. in a vacuum oven. The product (0.97 wt, 90%) was a light beige solid.

### EXAMPLE 3

### Preparation of ethyl 4,5-dihydrofaran-3-yl(oxo)acetate

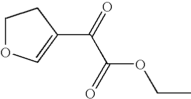

A flask was charged with 2,3-dihydrofuran (0.77 wt, 1.5 eq.), triethylamine (0.82 wt, 1.1 eq.) and MTBE (4 vol). To this solution at room temperature was added ethyl chlorooxoacetate (1 wt., 1 eq.) dropwise. During the addition the temperature rose and was kept below 35° C. by external cooling (total addition time 1 h). After the addition, the reaction was allowed to cool to room temperature, and stirred for 2 h. The reaction mixture was washed with water (3×2 vol.). The organic layer was concentrated at 30° C. to afford ethyl 4,5-dihydrofuran-3-yl(oxo)acetate as an oil (76-89%).

### EXAMPLE 4

### Preparation of (+/−)-1-(4,5-dihydrofuran-3-yl)ethane-1,2-diol

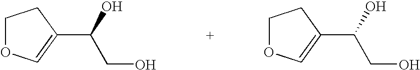

A flask fitted with an addition funnel, stirrer and nitrogen inlet was charged with lithium aluminum hydride (1M in THF, 5.8 vol, 1 eq.). The reaction was cooled to 15° C. A solution of ethyl 4,5-dihydrofuran-3-yl(oxo)acetate (1 wt., 1 eq.) in THF (4 vol) was added dropwise keeping the temperature between 15-20° C. (1 h total addition time). After the addition was complete, the reaction was stirred at room temperature for 30 min and then cooled to −5° C. A 2:1 solution of THF:water (I vol) was added slowly keeping the temperature below 5° C. A 15% sodium hydroxide solution (1 vol) was added dropwise, followed by water (0.3 vol). Celite (0.33 wt) was added and the resulting slurry was stirred 1 h at room temperature, filtered, and the filtercake was washed with tetrahydrofuran (total of 5 vol.) to produce a filtrate containing the title compound.

### EXAMPLE 5

### Preparation of rel-(3S,3aR)-3a-bromohexahydrofuro[2.3-b]furan-3-ol

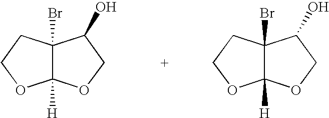 The solution of (+/−)-1-(4,5-dihydrofuran-3-yl)ethane-1,2-diol from Example 4 was cooled to ˜−10° C. and titrated with NBS (˜0.69 eq based on ethyl 4,5-dihydrofuran-3-yl(oxo)acetate used in example 4, ˜0.94 wt.). The titration was accomplished by monitoring the exotherm associated with the portionwise addition of NBS carried out over ˜1 h at ˜10° C. The reaction mixture was stirred for another ˜30 min and was treated with 10% sodium sulfite solution (6 vol.). The resulting solution (a 3:1 diastereomeric mixture) was concentrated to about one half the original volume in vacuo at 35-40° C. and extracted with methyl tert-butyl ether (5×5 vol.). The combined organics were washed with water (5×5 vol.), and concentrated to an oil to afford 3a-bromohexahydrofuro[2,3-b]furan-3-ol as a diastereomeric mixture of 95 to 5 (˜40% yield from ethyl 4,5-dihydrofuran-3-yl(oxo)acetate. The washes were combined and extracted with ethyl acetate (2×10 vol). The extracts, made up of a 3:1 mixture of diastereomers, were cycled through the above extraction process to isolate ˜10% additional product as a 95:5 diastereomeric mixture. Overall yield was ˜50% (two steps).

### EXAMPLE 6

### Preparation of rel-(3R,3aS,6aR)-hexahydrofuro[2,3-b]furan-3-yl acetate

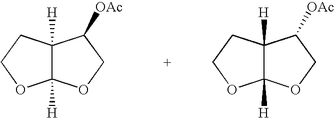 Rel-(3S,3aR,6aR)-3a-bromohexahydrofuro[2,3-b]furan-3-ol (1 wt., 1 eq) (95:5 mixture of diastereomers, unresolved), THF (4.2 wt.), and triethylamine (0.58 wt, 1.2 eq.) were charged to a reactor followed by a slurry of palladium on carbon (0.28 wt, 5% Pd/C, 50% water) in water (0.86 wt.). The mixture was subjected to hydrogen gas for ˜8 hours and the catalyst was removed by filtration and washed with THF (2×1 wt.). The resulting solution was concentrated to approximately half the volume and successively charged with ethyl acetate and concentrated to approximately half the volume to reduce water levels. Dichloromethane was charged (6.5 wt.) followed by triethylamine (0.58 wt., 1.2 eq.), and DMAP (0.005 wt., 0.01 eq.) and the mixture was cooled to ˜5° C. Acetic anhydride (0.58 wt., 1.2 eq.) was added over 30 min while keeping the temperature at 5-10° C. The reaction mixture was warmed to ˜23° C. over 1.5 h at which point the acetylation was complete. Methanol (0.076 wt., 0.50 eq.) was charged over 10 min while controlling the temperature at 23-27° C. After an additional 30 min of stirring, the organic layer was successively washed with water (2×2.5 wt.) and 3% HCl (aq) (2.5 wt.). The organic layer was concentrated to the title compound as a free flowing oil.

### EXAMPLE 7

### Preparation of 3R,3aS,6aR)-hexahydrofuro[2,3-b]furan-3-ol

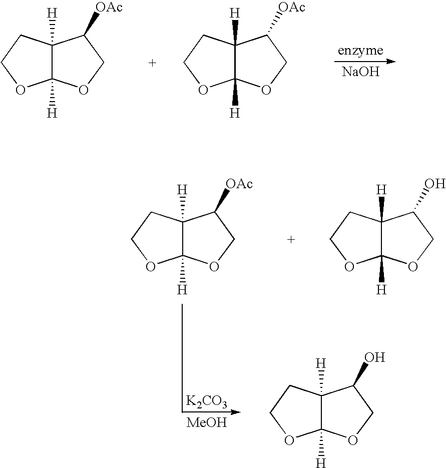 A reactor was charged with (racemic) rel-(3R,3aS,6aR)-hexahydrofuro[2,3-b]furan-3-yl acetate (1.0 eq) and 0.1N NaH₂PO₄ (pH=4, 3.0 vol). The resulting solution was adjusted to pH 5.0 with 15% aq NaOH. Altus ChiroClec-PC (5.10×10⁻⁴ wt) was added, the reaction heated to 40° C., and the pH kept between 4.8 and 5.2 with periodic addition of 15% aq NaOH. The reaction was followed by chiral GC until all of the undesired acetate [(3S,3aR,6aS)-hexahydrofuro[2,3-b]furan-3-yl acetate] had been hydrolyzed (˜3-5 h). The mixture was filtered through a small inline filter to remove the CLEC. The mixture was stirred for 15 min and allowed to settle. The layers were separated and the aqueous layer was extracted with DCM (2 vol). The organic layers were combined and wash with water (2×2 vol) until the undesired alcohol [(3S,3aR,6aS)-hexahydrofuro[2,3-b]furan-3-ol] was less than 3% in the organic layer. The resulting organic solution of the desired acetate [(3R,3aS,6aR)-hexahydrofuro[2,3-b]furan-3-yl acetate] was then concentrated at atmospheric to 1 vol. Methanol (2.0 vol) was added, followed by potassium carbonate (0.012 eq, 0.01 wt). The mixture was stirred for ˜2-3 hours whereupon conversion to the the desired alcohol [(3R,3aS,6aR)-hexahydrofuro[2,3-b]furan-3-ol)]was complete. Acetic acid (0.025 eq, 0.008 vol) was then added to neutralize the base and the mixture was concentrated at atmospheric and reduced pressure to ˜1 vol. Isopropyl acetate (2 vol) was added and the solution was again concentrated to ˜1 vol. Isopropyl acetate (2 vol) was again added and the solution was concentrated to 1 vol, and analyzed for MeOH content.

### EXAMPLE 8

### Preparation of (3R,3aS,6aR)-hexahydrofuro[2,3-b]furan-3-yl 4-nitrophenyl carbonate

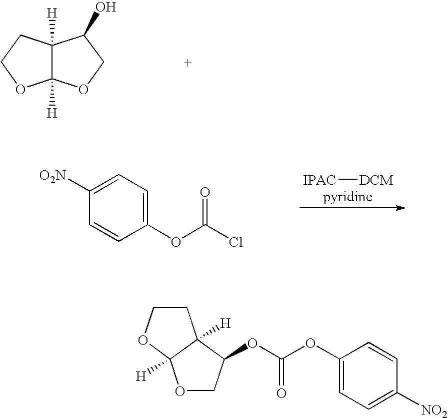 A flask was charged with a solution of (3R,3aS,6aR)-hexahydrofuro[2,3-b]furan-3-ol)(1.0 eq) from Example 7. Isopropyl Acetate was added to bring the total weight to 5.7 wt (˜5 vol). The reaction was cooled to 15° C. Pyridine (1.4 eq, 0.87 vol) was added producing a slight exotherm. The solution was cooled again to 15° C. A solution of 4-nitrophenyl chloroformate (1.3 eq, 2.02 wt) in dichloromethane (7 vol) was added as rapidly as possible while maintaining the reaction temperature 15 +/−3° C. An additional portion of dichloromethane (2 vol) was used in the transfer. Once the additon was complete, the reaction was warmed to 20° C. and stirred for 1 h. GC assay showed the level of residual (3S,3aR,6aS)-hexahydrofuro[2,3-b]furan-3-ol) was acceptable. HPLC assay indicated the extent of bis(4-nitrophenyl)carbonate formation was acceptable. Water (5 vol) was added and the layers were stirred 15 min, settled and separated. The organic phase was washed with 1N HCl (5 vol) and 5% aq sodium bicarbonate (5 vol). The organic solution was then concentrated at atmospheric to ˜5 vol. Isopropyl acetate (4 vol) was added, and the solution was cooled to 65° C. and seeded. The mixture was aged at 65° C. for 30 min and the resultant slurry was then cooled to 0° C. over 1 h, and aged for 1 h. Filtration with an MTBE wash (2 vol) afforded a light powder. The material was assayed by HPLC to demonstrate that the level of bis(4-nitrophenyl)carbonate was less than 2%. The title product was dried in vacuo at 40-60-C (70-78%).

### EXAMPLE 9

### Preparation of tert-butyl (1S,2R)-3-[(1,3-benzodioxol-5-ylsulfonyl)(isobutyl)amino]-2-hydroxy-1-{4-[(2-methyl-1,3-thiazol-4-yl)methoxy]benzyl}propylcarbamate

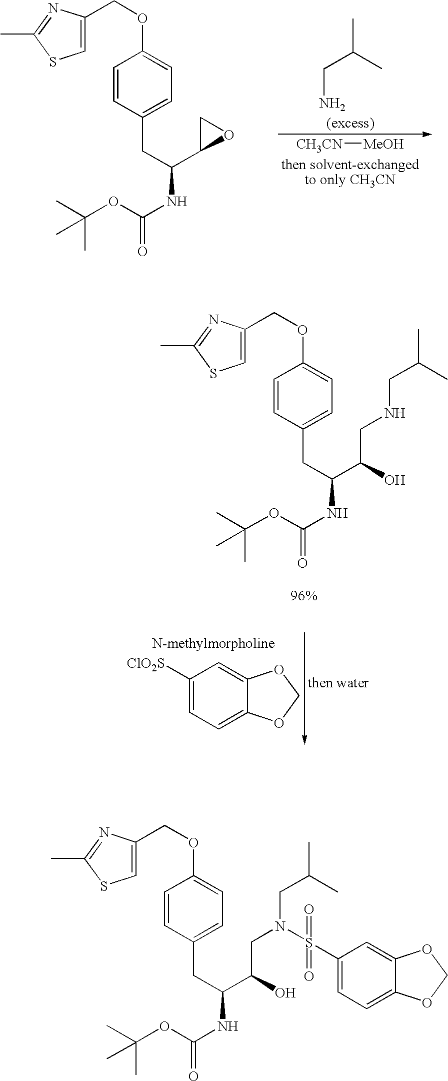

A reaction vessel was charged with tert-butyl (1S)-2-{4-[(2-methyl-1,3-thiazol-4-yl)methoxy]phenyl}-1-[(2S)-oxiran-2-yl]ethylcarbamate (1.0 wt.) followed by acetonitrile (3.5 vol.), methanol (1.0 vol.), and isobutylamine (8.3 equiv., 2.1 vol.). The resulting mixture was heated to reflux and held at reflux for 3 h. Acetonitrile (6.0 vol.) was charged and distillate (6.0 vol., 4.6 wt.) was collected at atmospheric pressure. A second portion of acetonitrile (6.0 vol.) was charged and distillate (7.0 vol., 5.5 wt.) was collected at atmospheric pressure. Acetonitrile (2.0 vol.) and N-methylmorpholine (1.3 eq, 0.37 vol) was charged and the reaction mixture was cooled to ˜25° C. A solution of 1,3-benzodioxole-5-sulfonyl chloride (1.1 equiv., 0.62 wt) was charged while maintaining a temperature of ˜25° C. The resulting reaction mixture was stirred for 4 h. The solution was heated to 65° C. and water (2.5 vol) was charged maintaining 65° C. The solution was seeded with tert-butyl (1S,2R)-3-[(1,3-benzodioxol-5-ylsulfonyl)(isobutyl)amino]-2-hydroxy-1-{4-[(2-methyl-1,3-thiazol-4-yl)methoxy]benzyl}propylcarbamate (0.01 wt) and aged for 0.5 h at 65° C., wherupon water (5 vol) was charged again, maintaining the temperature at 65 IC. The resulting slurry is cooled to 25° C. and stirred for 1 h. The solids are filtered and washed with a solution of acetonitrile (0.5 vol.) in water (1.5 vol). The product was dried at ˜50° C. in a vacuum oven. The title product (1.6 wt, 95%) was a light beige solid.

### EXAMPLE 10

### Preparation of N-(3R, 3aS, 6aR)-hexahydrofuro[2,3-b]furan-3-yl-oxycarbonyl-, (4S,5R)4-[4-(2-methylthiazolo-4-methyloxy)-benzyl]-5-1-butyl-[(3,4-methylenedioxyphenyl)sulfonyl]-aminomethyl-2,2-dimethyl-oxazolidine

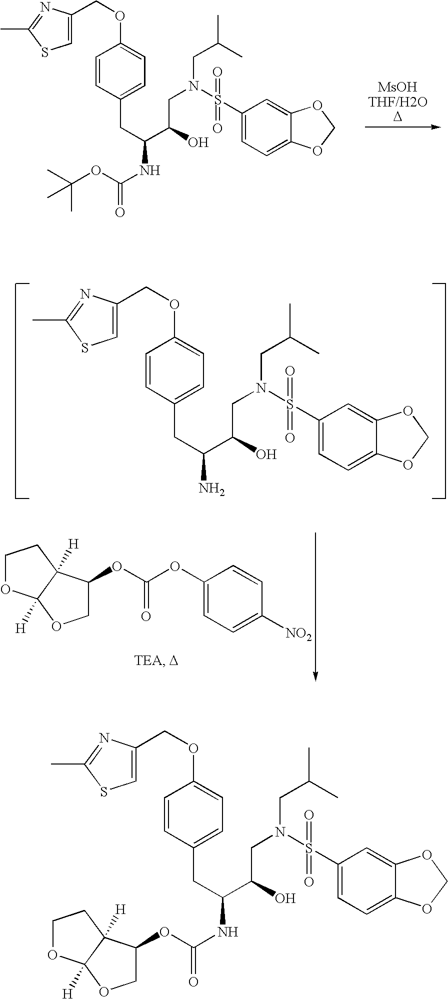 A reaction vessel was charged with tert-butyl (1S,2R)-3-[(1,3-benzodioxol-5-ylsulfonyl)(isobutyl)amino]-2-hydroxy-1-{4-[(2-methyl-1,3-thiazol-4-yl)methoxy]benzyl}propylcarbamate (1.0 wt.), tetrahydrofuran (5.0 vol.), and water (0.3 vol.) and stirred at ˜25° C. The reaction vessel was then charged with methane sulfonic acid (3.0 equiv., 0.30 vol.), and the resulting mixture was heated over 30 min to ˜50° C., stirred, and then heated over 30 min to reflux. The reaction mixture was cooled to ˜50° C., and triethylamine (3.7 equiv., 0.80 vol.) was added followed by solid (3R,3aS,6aR)-hexahydrofuro[2,3-b]furan-3-yl 4-nitrophenyl carbonate. (1.05 equiv., 0.48 wt.). The resulting mixture was brought to reflux, and stirred for 3.5 h. The reaction mixture was cooled to ˜50° C. and tert-butyl methyl ether (3.0 vol.) was added. Maintaining ˜50° C., the mixture was washed with water (2.1 vol.), 10% (by weight) aqueous potassium carbonate (2×2.1 vol., 1.0 equiv) and 5% (by weight) aqueous acetic acid (2.1 vol., 1.1 equiv) At atmospheric pressure, the organic mixture was concentrated to ˜4.2 vol (i.e. ˜3.3 vol removed) and diluted to the original volume with isopropyl alcohol (˜3.3 vol.) The mixture was again taken to 4.2 vol and diluted to the original volume with isopropyl alcohol (˜3.3 vol.). After a final concentration to 4.2 vol, isopropyl alcohol (9.5 vol.) and water (0.30 vol) was charged. The resulting mixture heated to dissolve all solids, cooled to 50° C., seeded with N-(3R, 3aS, 6aR)-hexahydrofuro[2,3-b]furan-3-yl-oxycarbonyl-, (4S,5R)4-[4-(2-methylthiazolo-4-methyloxy)-benzyl]-5-1-butyl-[(3,4-methylenedioxyphenyl)sulfonyl]-aminomethyl-2,2-dimethyl-oxazolidine crystals (0.01 wt), and held at 50° C. for 0.5 h (or until appreciable crystallization has occurred. The resulting slurry was cooled to 35° C. at 0.5° C./min (˜30 min) and stirred for 1 h. The slurry was further cooled to 0° C. at 1.0° C./min (˜35 min) and stirred for 1 h. The solid product was filtered, washed with cold isopropyl alcohol 2×2 vol.), and dried at ˜65° C. in a vacuum oven. The product (0.97 wt, 90%) was a light beige solid.

### EXAMPLE 11

### Preparation of (3R,3aS,6aR)-hexahydrofuro[2,3-b]furan-3-yl-4-carbomethoxycarbonate

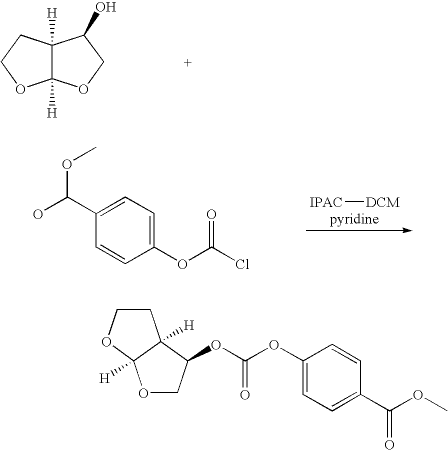 A flask was charged with a solution of 4-carbomethoxyphenyl chloroformate (1.2 eq) in ethyl acetate (6 vol) and cooled with stirring to 5° C. A solution of (3R,3aS,6aR)-hexahydrofuro[2,3-b]furan-3-ol)(1.0 eq) from Example 7, pyridine (1.4 eq) and ethyl acetate (4 vol) was added slowly maintaining the temperature below 15° C. The reaction was warmed to room temperature and stirred for 1.5 h while following by GC. When the reaction was deemed complete, it was quenched with water (4 vol) and heated to 35° C. The layers were separated and the organic washed with 1 NHCl (2×1 vol) and brine (1 vol). The solution was dried by adding ethyl acetate (2 vol) distilling off solvent (2 vol) at atmospheric. The solution was then cooled to room temperature and filtered to remove bis-(4-carbomethoxyphenyl)-carbonate. The filtrate was concentrated further and the title compound crystallized using heptane as an anti-solvent. The slurry was filtered, washing the cake with heptane (2 vol). The cake was dried under vacuum at ambient. The title compound was obtained as a beige solid.

### EXAMPLE 12

### Preparation of (3R,3aS,6aR)-hexahydrofuro[2,3-b]furan-3-yl-1N-benzotriazole carbamate

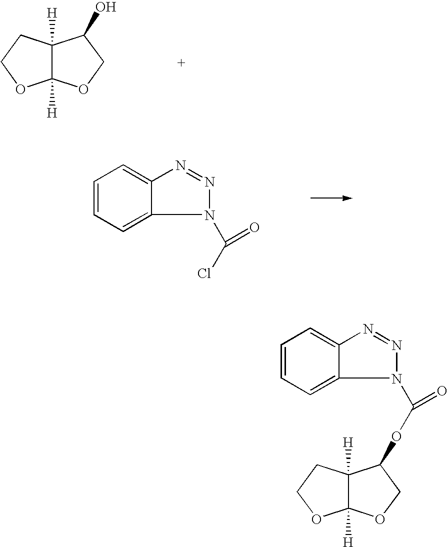

A flask was charged with 1H-1,2,3-benzotriazole-1-carbonyl chloride (1.2 eq) and dimethoxymethane. A solution of (3R,3aS,6aR)-hexahydrofuro[2,3-b]furan-3-ol)(1.0 eq) from Example 7, N-methylmorpholine (1.3 eq), and dimethoxyethane (0.25 vol) was added slowly, with stirring, maintaining the temperature at 40° C. The reaction was then aged at 40° C. for 1.5 h while following by GC. Once the reaction was deemed complete, it was cooled to 30° C. and water (2.5 vol) was added. The solution was cooled to 20° C. slowly and crystallization occurred. Added water (3.5 vol) slowly, cooled to 15° C., and stirred for 10 min. The slurry was filtered, washing the cake with water (2.5 vol). The cake was then dried in the oven under vacuum at 60° C. (3R,3aS,6R)-hexahydrofuro[2,3-b]furan-3-yl-1N-benztrazole carbamate was obtained as a beige solid.

1. A process for the preparation of a compound of formula (I) 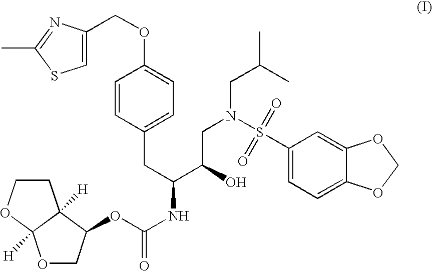
comprising:
(a) treating a compound of formula (II) 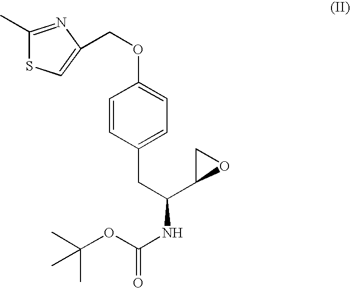
with excess isobutylamine in an alcohol-containing solvent to form a compound of formula (III) 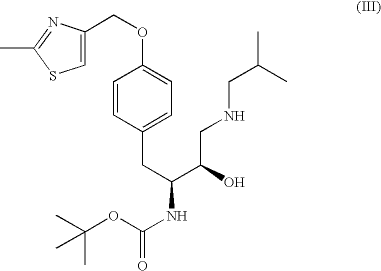
(b) treating a compound of formula (III) with a compound of formula (IV) 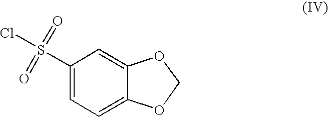
in the presence of an aqueous base to form a compound of formula (V) 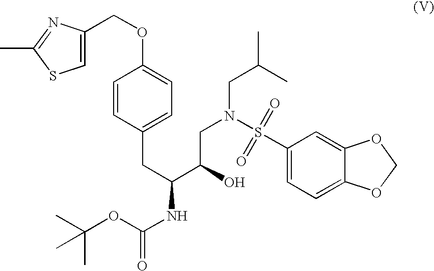
(c) deprotecting a compound of formula (V) to form a compound of formula (VI) 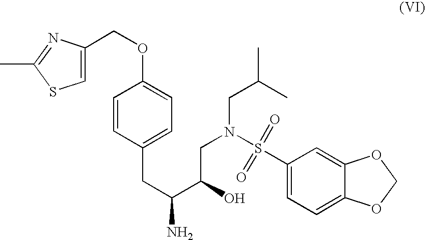
(d) coupling a compound of formula (VI) with a compound of formula (VII) 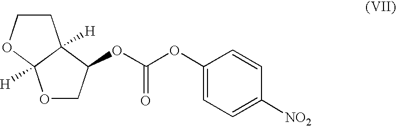
to yield a compound of formula (I).

2. A a process for the preparation of a compound of formula (I) comprising steps (a), (b), (c) and (d) according to claim 1 wherein steps (a) and (b) are combined in a one-pot reaction to yield a compound of formula (V) which is isolated and in which steps (c) and (d) are combined in a one-pot reaction to yield a compound of formula (I).

3. A process for the preparation of a compound of formula (I) 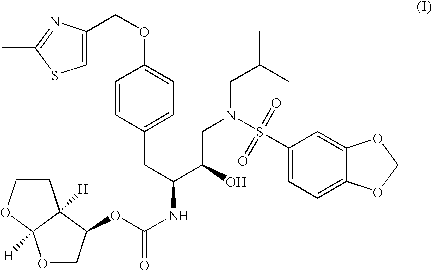
comprising:
(a) treating a compound of formula (II) 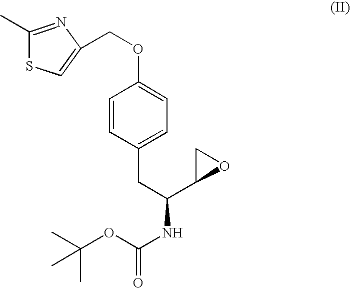
with excess isobutylamine in an alcohol-containing solvent to form a compound of formula (III); 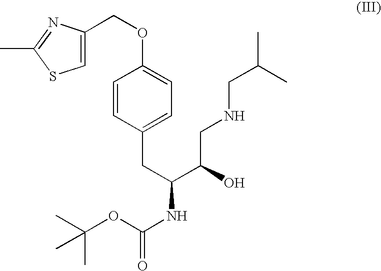
(b) treating a compound of formula (III) with a compound of formula (IV) 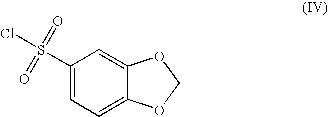
in the presence of an aqueous base to form a compound of formula (V) 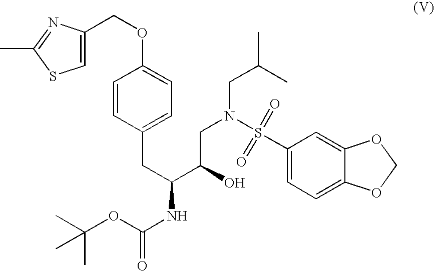
(c) deprotecting a compound of formula (V) and coupling with a compound of formula (VII) 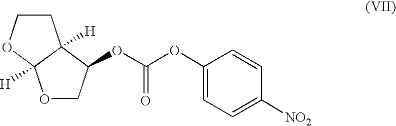
to form a compound of formula (I).

4. A process for the preparation of a compound of formula (I) 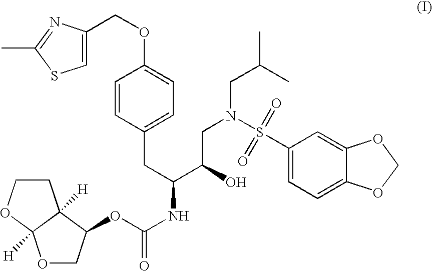
comprising:
(a) treating a compound of formula (II) 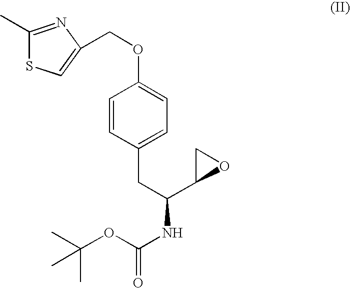
with excess isobutylamine in an alcohol-containing solvent to form a compound of formula (III) 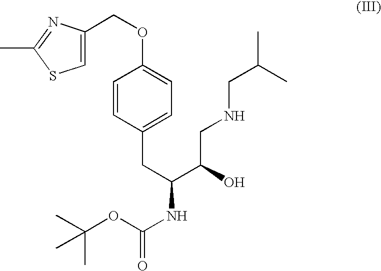
(b) treating a compound of formula (III) with a compound of formula (IV) 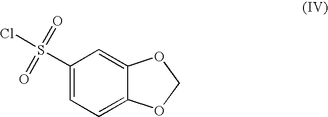
in the presence of an aqueous base to form a compound of formula (V)> 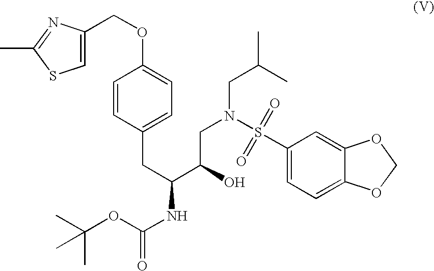
(c) deprotecting a compound of formula (V) to form a compound of formula (VI) 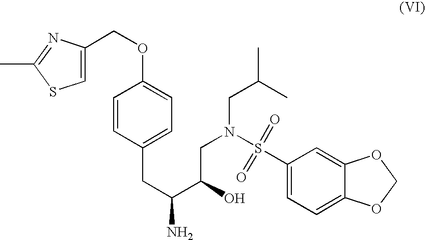
(d) coupling a compound of formula (VI) with a compound of formula (VI) 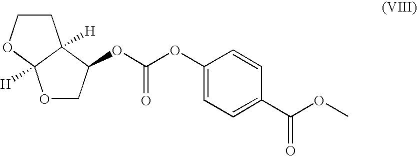
to yield a compound of formula (I).

5. A process for the preparation of a compound of formula (I) comprising steps (a), (b), (c) and (d) according to claim 4 wherein steps (a) and (b) are combined in a one-pot reaction to yield a compound of formula (V) which is isolated and in which steps (c) and (d) are combined in a one-pot reaction to yield a compound of formula (I).

6. A process for the preparation of a compound of formula (I) 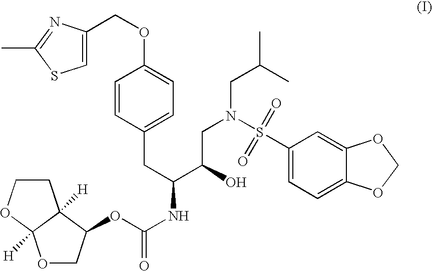
comprising:
(a) treating a compound of formula (II) 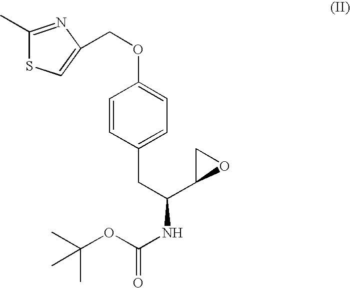
with excess isobutylamine in an alcohol-containing solvent to form a compound of formula (III) 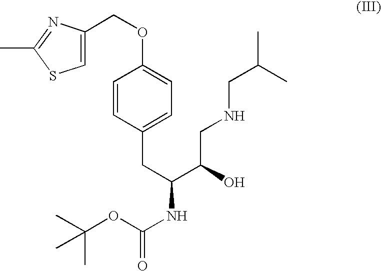
(b) treating a compound of formula (III) with a compound of formula (IV) 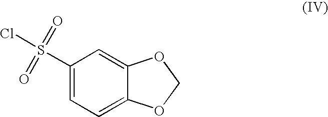
in the presence of an aqueous base to form a compound of formula (V) 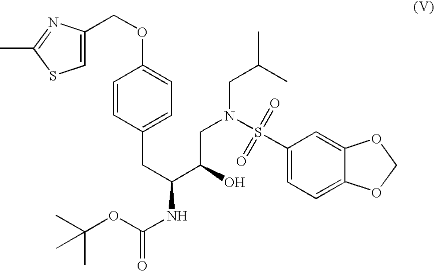
(c) deprotecting a compound of formula (V) and coupling with a compound of formula (VIII) 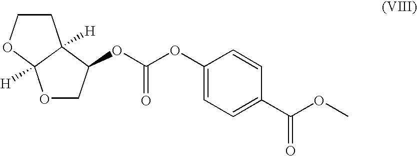
to form a compound of formula (I).

7. A process for the preparation of a compound of formula (I) 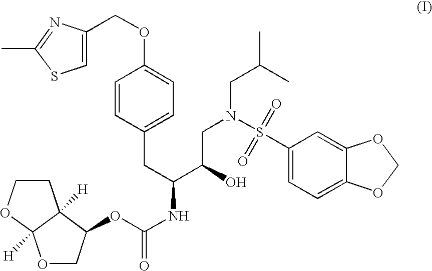
comprising:
(a) treating a compound of formula (II) 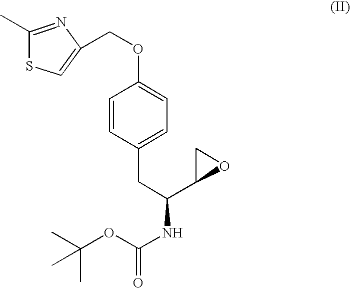
with excess isobutylamine in an alcohol-containing solvent to form a compound of formula (III) 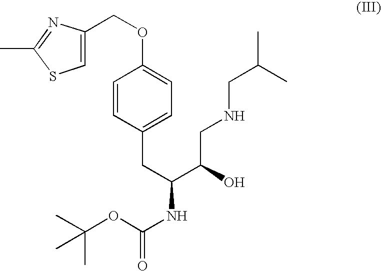
(b) treating a compound of formula (III) with a compound of formula (IV) 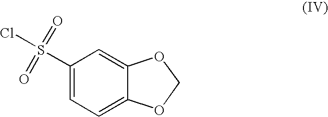
in the presence of an aqueous base to form a compound of formula (V) 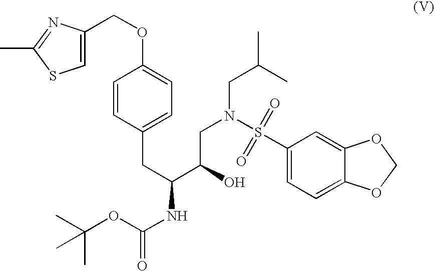
(c) deprotecting a compound of formula (V) to form a compound of formula (VI) 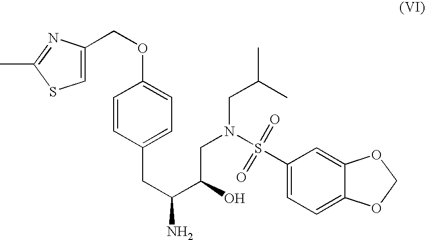
(d) coupling a compound of formula (VI) with a compound of formula (IX) 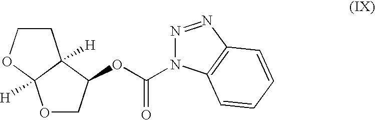
to yield a compound of formula (I).

10. A process for the preparation of a compound of formula (I) comprising steps (a), (b), (c) and (d) according to claim 7 wherein steps (a) and (b) are combined in a one-pot reaction to yield a compound of formula (V) which is isolated and in which steps (c) and (d) are combined in a one-pot reaction to yield a compound of formula (I).

11. A process for the preparation of a compound of formula (I) 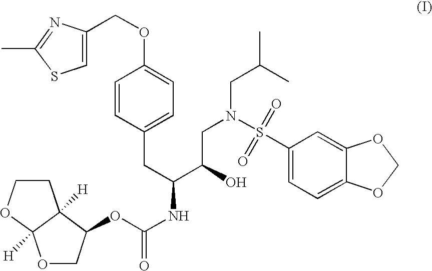
comprising:
(a) treating a compound of formula (II) 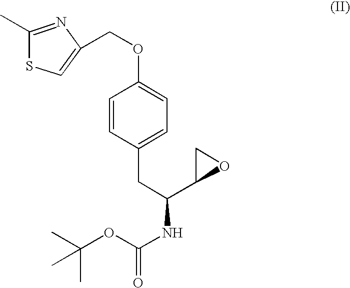
with excess isobutylamine in an alcohol-containing solvent to form a compound of formula (III) 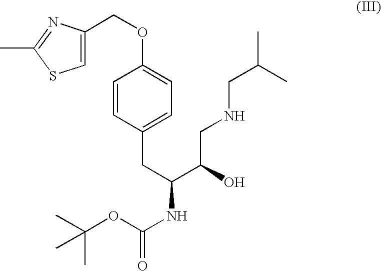
(b) treating a compound of formula (III) with a compound of formula (IV) 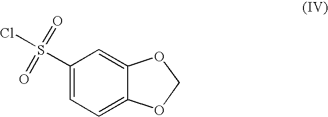
in the presence of an aqueous base to form a compound of formula (V) 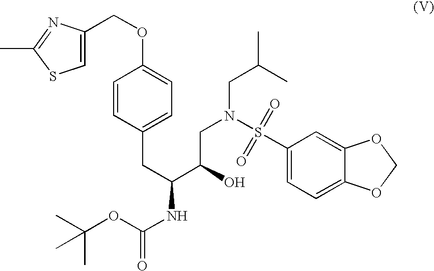
(c) deprotecting a compound of formula (V) and coupling with a compound of formula (IX) 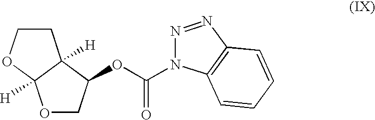
to form a compound of formula (I).

12. A process according to claim 7 wherein the alcohol-containing solvent is acetonitrile-methanol.

13. A process according to claim 7 wherein the aqueous base is sodium bicarbonate.

14. A process according to claim 4 wherein step (b) is performed in the presence of non-aqueous base.

15. A process for the preparation of (3R,3aS,6aR)-hexahydrofuro[2,3-b]furan-3-yl 4-nitrophenyl carbonate of the formula 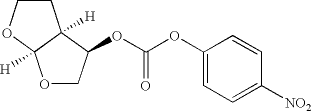
comprising reacting (3S,3aR,6aS)-hexahydrofuro[2,3-b]furan-3-ol) of the formula 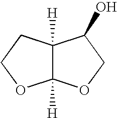
with 4-nitrophenyl chloroformate in a suitable solvent to form (3R,3aS,6aR)-hexahydrofuro[2,3-b]furan-3-yl 4-nitrophenyl carbonate.

16. (3R,3aS,6aR)-hexahydrofuro[2,3-b]furan-3-yl 4-nitrophenyl carbonate of the formula 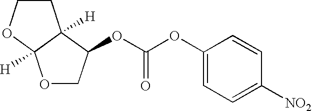
made by the process according to claim 15.

17. A compound of formula (VIII)

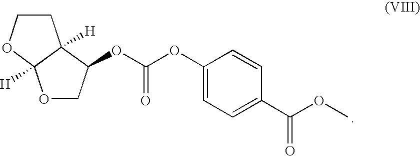

18. A compound of formula (IX)

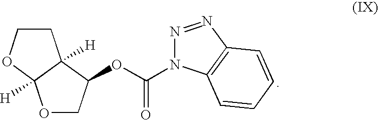

19. A process for the preparation of a compound of formula (I) 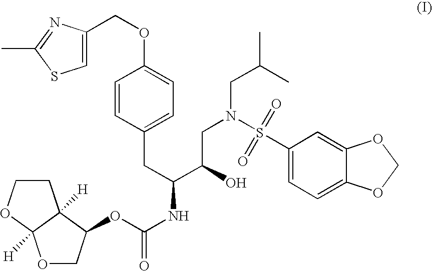
comprising:
(a) treating a compound of formula (II) 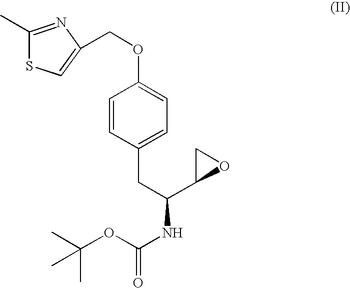
with excess isobutylamine in an alcohol-containing solvent to form a compound of formula (III) 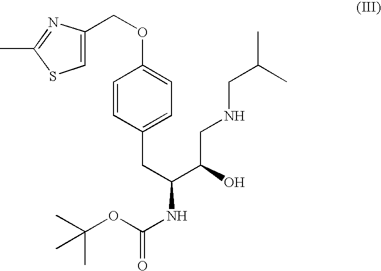
(b) treating a compound of formula (III) with a compound of formula (IV) 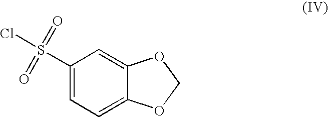
in the presence of non-aqueous base to form a compound of formula (V) 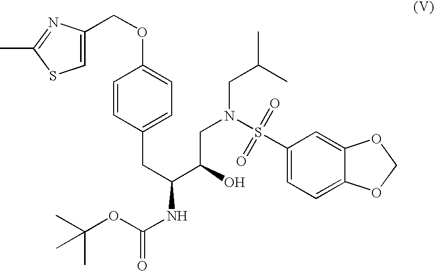
(c) deprotecting a compound of formula (V) to form a compound of formula (VI) 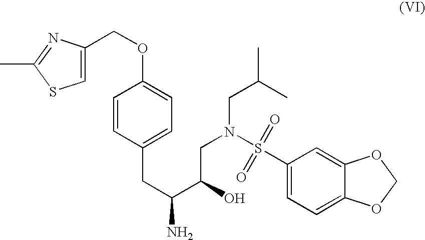
(d) coupling a compound of formula (VI) with a compound of formula (IX) 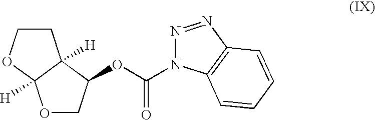
to yield a compound of formula (I).

20. A process according to claim 19 wherein the non-aqueous base is N-methylmorpholine.

21. A process for the preparation of a compound of formula (I) 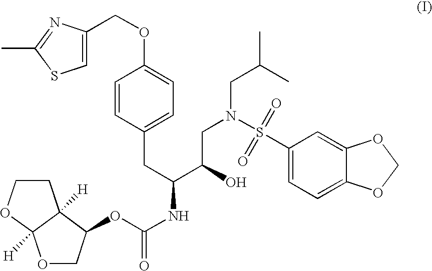
comprising:
(a) treating a compound of formula (II) 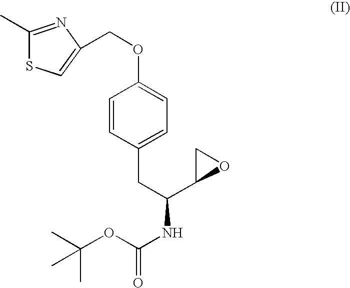
with excess isobutylamine in an alcohol-containing solvent to form a compound of formula (III) 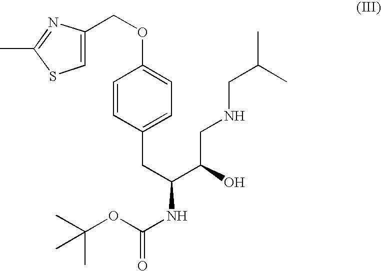
(b) treating a compound of formula (III) with a compound of formula (IV) 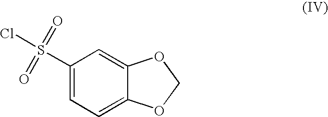
in the presence of a non-aqueous base to form a compound of formula (V) 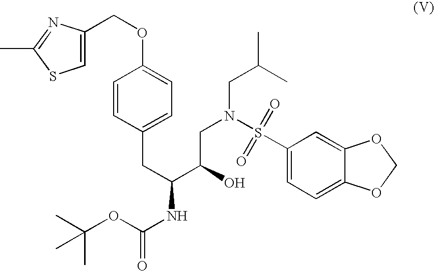
(c) deprotecting a compound of formula (V) to form a compound of formula (VI) 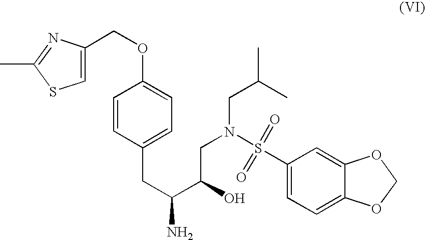
(d) coupling a compound of formula (VI) with a compound of formula (VII) 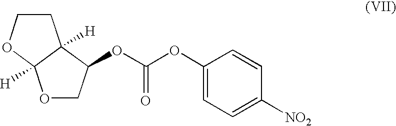
to yield a compound of formula (I).

22. A process according to claim 20 wherein the non-aqueous base is N-methylmorpholine.

23. A process according to claim 1 wherein the alcohol-containing solvent is acetonitrile-methanol.

24. A process according to claim 1 wherein the aqueous base is sodium bicarbonate.
